(19) United States
(12) Patent Application Publication
(10) Pub. No.: US 2026/0062658 A1
(43) Pub. Date: Mar. 5, 2026

(54) A MODULAR BIOPROCESSING SYSTEM

(71) Applicant: Oribiotech LTD, London, Greater London (GB)

(72) Inventors: Farlan Singh Veraitch, Greater London, London (GB); Stephen Shapka, London, Greater London (GB); John Cattermole, London, Greater London (GB); Nicholas Warrington, London, Greater London (GB); Andrew Clark, London, Greater London (GB)

(21) Appl. No.: 19/104,988

(22) PCT Filed: Aug. 17, 2023
(86) PCT No.: PCT/GB2023/052162

§ 371 Date: Feb. 19, 2025

(30) Foreign Application Priority Data
Aug. 19, 2022 (GB) 2212095.0

(51) Int. Cl. C12M 1/00; C12M 1/34; C12M 3/00; G01N 35/00

(52) U.S. Cl. CPC C12M 23/50; C12M 23/26; C12M 23/44; C12M 23/48; C12M 23/58; C12M 41/14; C12M 41/36; G01N 35/0099; G01N 2035/00326

(57) ABSTRACT

A modular bioprocessing system having a plurality of bioprocessing modules, each independently receiving a biological handling container and performing one or more unit operations on the biological handling container and/or its contents; and a robot that moves the biological handling container between each of the bioprocessing modules.

### CROSS-REFERENCE TO RELATED APPLICATIONS

This application is a national phase entry under 35 U.S.C. § 371 of International Patent Application PCT/GB2023/052162, filed Aug. 17, 2023, designating the United States of America and published as International Patent Publication WO 2024/038280 A1 on Feb. 22, 2024, which claims the benefit under Article 8 of the Patent Cooperation Treaty of United Kingdom Patent Application Serial No. 2212095.0, filed Aug. 19, 2022.

### TECHNICAL FIELD

This disclosure relates to a modular bioprocessing system and a method of processing material, such as biological material.

### BACKGROUND

The process of manufacturing a bioproduct at scale is expensive due to the large amount of human labor required for each unit operation. In particular, in a cell and/or gene therapeutic manufacturing process, multiple unit operations are carried out in various systems that lack synergy between them, hence adding complexity to the process, which results in an increased cost for therapeutic manufacturers. As a result, access to therapies are limited to the vast majority of patients. Therefore, an affordable cell-based research system and/or a therapeutic manufacturing system, is becoming increasingly important to patients.

More recently, there has been a push to move toward automated cell-based research systems and/or therapeutic manufacturing systems. In some examples, automated systems are provided whereby entire modules and equipment are moved between various locations of the system for appropriate processing. However, such systems suffer many drawbacks. For example, such systems require complex and mechanically robust robotic mechanisms to enable such heavy equipment to be transported between locations, which are often expensive. Additionally, such systems are difficult to incorporate either new or existing processing technologies, as would be required by an end user from time-to-time, owing to the fact that a new module would have to be created in its entirety, rather than simply incorporating off-the-shelf equipment directly into the system.

Therefore, it is an object of the disclosure to provide a scalable, automated and cost-effective bioprocessing system, particularly one in which biological therapeutics, such as a cell and/or gene therapeutic product, can be manufactured.

Moreover, it is a further object of the disclosure to provide a scalable and automated bioprocessing system, which can be automated cost effectively and/or allow for compatibility with new or existing off-the-shelf technologies.

### BRIEF SUMMARY

In accordance with one aspect of the disclosure, there is provided a modular bioprocessing system, comprising a plurality of bioprocessing modules, each being independently configured to receive at least one biological handling container and perform one or more unit operations thereon and/or on contents therein; and a robot configured and arranged to move the, or each, biological handling container between each of the plurality of bioprocessing modules.

That is, there are a number, i.e., more than one, bioprocessing modules, each of which are able to receive one or more biological handling containers. Each bioprocessing module may have a portion, such as a holder, a gripper, a mounting plate, a drawer, a stand or the like, for receiving one or more biological handling containers. Alternatively or additionally, each bioprocessing module may have a portion, such as an actuator, a plunger, a gripper, a sensor, or the like, that allows one or more unit operations to be performed on the biological handling container itself, on the contents within the biological handling container, or both on the biological handling container itself and the contents within the biological handling container. Particularly, each bioprocessing module may include an actuator configured to actuate a connector, such as a needle-based connector, coupled to the biological handling container. Alternatively or in addition, the actuator may be configured to actuate the biological handling container so as to cause dispensation of the contents of the biological handling container. Particularly, the actuator may be configured to impart a force, such as a compressive force, onto at least a portion of the biological handling container, where at least a portion of the biological handling container is flexible and/or compressible. The actuator(s) configured for actuation of a connector and/or a biological handling container may be formed as a single actuator or separate, multiple, actuators.

That is, there is provided a robot that moves the one or more biological handling container(s) between each of the bioprocessing modules. The robot may include a robotic arm, a gripper or the like that enables the robot to receive or collect the biological handling container(s) from one bioprocessing module. The robot may also be provided on rails, wheels or the like to enable movement between bioprocessing modules. The robot may also include an actuator configured to actuate a portion of the biological handling container. The actuator may be configured to actuate a connector, such as a needle-based connector, coupled to the biological handling container. Alternatively or in addition, the actuator may be configured to actuate the biological handling container so as to cause dispensation of the contents of the biological handling container. Particularly, the actuator may be configured to impart a force, such as a compressive force, onto at least a portion of the biological handling container, where at least a portion of the biological handling container is flexible and/or compressible. The actuator(s) configured for actuation of a connector and/or a biological handling container may be formed as a single actuator or separate, multiple, actuators.

As used herein, the term “bioprocessing system” is intended to refer to a system in which one or more operations in the manufacture of a bioproduct, such as a cellular product, can take place. The term “bioproduct manufacturing system” may be synonymously used. Such operations include, but are not limited to, the analysis, preparation, processing, storage, modification, manipulation, culturing, mixing, dilution, harvesting or the like of any suitable material used in the manufacture of a bioproduct, such as a cellular product. The material may include a medium for cells, with or without cells therein, a growth factor, a virus, a bead-based reagent, a final product such as a final bioproduct, or the like. The cellular product may be a CAR-T product.

As used herein, the term “biological processing module” is intended to refer to a module in which one or more specific operations in the manufacture of a bioproduct, such as a cellular product, can take place. In some examples, each biological processing module is static, or immobile. That is, they are not moved by, or capable of being moved by, the robot, or other alternative means. That is, each biological processing module is static and the robot is moveable with respect to each biological processing module. Each biological processing module may include an apparatus or apparatuses that enable the one or more specific operations to take place. The apparatus or apparatuses, which enable the one or more specific operations to take place, may remain static or immobile within their respective biological processing module. That is, they are not moved by, or capable of being moved by, the robot, or other alternative means.

As used herein, the term “biological handling container” is intended to refer to a container that is suitable for handling biological material or material used in the manufacture of a bioproduct. In some examples, the biological handling container may be a bioreactor, a consumable, an auxiliary container, a sampling container, a vacutainer or the like. In some examples, the biological handling container may include an interfacing element, such as an interfacing plate, to provide fluid communication to the biological handling container. In particular examples, the biological handling container comprises at least a portion that is flexible and/or compressible, or the entirety of the biological handling container is compressible and/or flexible. In some examples, the biological handling container may be coupled to a connector, such as a needle-based connector. The needle-based connector may be that as described in WO2021/123760 A1.

Advantageously, the bioprocessing system described herein can produce a bioproduct with minimal human intervention. Further advantageously, the bioprocessing system described herein can produce a bioproduct in a semi, or fully, automated manner. Yet further advantageously, materials can be provided to a relevant bioprocessing module in a “just-in-time” manner, i.e., the relevant biological handling container is only transported to the relevant bioprocessing module when deemed required by a user or by the system. Moreover, by moving the biological handling container—and not the biological processing module per se—the bioprocessing system described herein is more suited to incorporating new and existing off-the-shelf technologies, as would be required by the end user.

Optionally, the modular bioprocessing system comprises the biological handling container. In such a case, there is provided a modular bioprocessing system, comprising a biological handling container, a plurality of bioprocessing modules, each being independently configured to receive the biological handling container and perform one or more unit operations thereon and/or on contents therein; and a robot configured to move the biological handling container between each of the plurality of bioprocessing modules.

Optionally, at least one of the plurality of bioprocessing modules is a bioreactor module configured to maintain suitable conditions for cell culture. That is, a bioreactor module is configured to receive a biological handling container, such as a bioreactor, and enabling a cell culture process to be carried out therein, under suitable conditions to maintain such a cell culture. Optionally, the bioreactor module comprises a bioreactor housed therein.

Optionally, the bioreactor module comprises a first dock that is configured to receive a first biological handling container, and a second dock that is configured to receive a second biological handling container. Particularly, the first biological handling container may be a bioreactor, and the second biological handling container may be an auxiliary container, with or without a connector coupled thereto.

Optionally, the bioreactor module comprises an incubator configured to substantially maintain an internal atmosphere and/or temperature. In some examples, the internal atmosphere may include a carbon dioxide content of approximately 5% to approximately 10%, preferably approximately 5%. In some examples, the temperature may be approximately 37 degrees Celsius.

Optionally, at least one of the plurality of bioprocessing modules is a biological analysis module. That is, a biological analysis module is configured to receive a biological handling container and perform one or more analyses, on the biological handling container per se and/or on the contents thereof. The analysis may be performed on the biological handling container to confirm the sterility, integrity, or other mechanical properties of the biological handling container. Alternatively or in addition, the analysis may be performed on the contents of the biological handling container, either directly, in which case a sample of the contents is taken from the biological handling container, or indirectly, in which case the contents remain within the biological handling container.

Optionally, the biological analysis module comprises at least one of: a pH meter, a cell counter, a cell seeding density meter, a flow cytometer, a polymerase chain reaction device, a sterility analyzer, a media analyzer, a metabolite analyzer, a cell imaging device and a microscope.

Optionally, at least one of the plurality of bioprocessing modules is a preparation module. That is, a preparation module is configured to receive a biological handling container and prepare contents for dispensing into the biological handling container. Optionally, the preparation module includes a dispensation device configured to dispense a fluid into the biological handling container. The fluid may include liquid, gas, slurries, suspensions, gels and other fluid-like materials. Optionally, the preparation module includes a plurality of reservoirs, each containing a fluid, a mixing device for optionally mixing the fluids, and a dispensation device for dispensing the fluid, or mixed fluid, into the biological handling container.

Optionally, the preparation module is configured to prepare and/or process at least one of: cell culture media, growth factors, viral vectors, non-viral vectors and bead-based reagents.

Optionally, the preparation module is configured to dispense at least one of: cell culture media, growth factors, viral vectors, non-viral vectors and bead-based reagents into the biological handling container.

Optionally, at least one of the plurality of bioprocessing modules is a cell selection module, Particularly, the cell selection module may be a magnetic-activated cell selection module. In particular, the cell section module may comprise a selectively magnetizable device configured to select, i.e., positively or negatively, cells from a sample. Any such selectively magnetizable device is suitable for such a module.

Optionally, at least one of the plurality of bioprocessing modules is a centrifuge module. That is, a centrifuge module may include a centrifuge configured to receive the biological handling container for centrifuging the contents thereof.

Optionally, at least one of the plurality of bioprocessing modules is a washing module. That is, a washing module may include an apparatus configured to receive the biological handling container and to wash the contents (such as a cell culture) thereof. In particular, the washing module may comprise a centrifuge, a counterflow centrifugation elutriation (CCE) device, a filtration device, or the like.

Optionally, at least one of the plurality of bioprocessing modules is an electroporation module. That is, an electroporation module may include an electroporation device configured to apply an electric field to the contents of the biological handling device.

Optionally, at least one of the plurality of bioprocessing modules is a cell harvesting module. That is, a cell harvesting module may include a device for harvesting cells—i.e., the removal of cells from the remainder of the contents within the biological handling container. The device may comprise a cell harvesting device and a container, such as a flexible bag, for receiving the harvested cells.

Optionally, at least one of the plurality of bioprocessing modules is a formulation module. That is, a formulation module may include a device for formulating the cells harvested from the biological handling container into a final formulation for administration to a patient. The device may comprise a container, such as a flexible bag, configured to receive (or including) harvested cells, and one or more further containers each configured to receive (or including) buffers, pharmaceutically acceptable excipients or media, and like reagents to enable the final formulation of a cell therapy product for administration to a patient. The device may also comprise means to enable mixing and/or dispensation of the harvested cells and the buffers, pharmaceutically acceptable excipients or media, and other like substances so as to provide a final formulation in a final formulation container. The final formulation container may be a flexible bag, such as an intravenous (IV) bag.

Optionally, at least one of the plurality of bioprocessing modules is a storage module. That is, a storage module may include a storage device, such as an incubator, a cupboard, a fridge or a freezer. The storage module may be configured to maintain a temperature of approximately 4 degrees Celsius. In such an example, the storage module may be a media storage module, i.e., configured to maintain a biological handling container including media therein at a temperature of approximately 4 degrees Celsius. The storage module may be configured to maintain a temperature of approximately −20 degrees Celsius. In such an example, the storage module may be a growth factor, or DNA, storage module, i.e., configured to maintain a biological handling container including growth factors and/or DNA therein at a temperature of approximately −20 degrees Celsius. The storage module may be configured to maintain a temperature of approximately −80 degrees Celsius. In such an example, the storage module may be a virus storage module, i.e., configured to maintain a biological handling container including virus therein at a temperature of approximately −80 degrees Celsius. In some examples, the storage module may include various compartments, each being configured to maintain a different temperature, such as those noted herein. In some examples, the storage module may be configured to freeze or thaw contents of a biological handling container.

Optionally, at least one of the plurality of bioprocessing modules comprises a device for reading a designation feature on the biological handling container. That is, there may be provided a device, such as a scanner, for detecting and reading a feature disposed on the biological handling container.

Optionally, the designation feature is one or more of an RFID tag, an NFC tag, a barcode or a QR code.

Optionally, the modular bioprocessing system further comprises a microprocessor configured to: receive a signal from at least one of the plurality of bioprocessing modules; and in response to receiving the signal from at least one of the plurality of bioprocessing modules, generate and send a signal to the robot such that the robot is caused to move the biological handling container from a first bioprocessing module to a second bioprocessing module.

Optionally, each bioprocessing module is formed as a compartment. In some examples, the compartment is formed having a substantially cuboidal volume.

Optionally, the bioprocessing system comprises a first side and a second side, each compartment formed on the first side and a maintenance portion formed on the second side, the maintenance portion comprising at least one of: a fluid connector, a fluid reservoir, an electrical connector, a gas connector, a gas source, a computer, a network adaptor and a control panel.

Optionally, an interior of each compartment is isolated from an interior of another compartment. Optionally, the internal environment of each compartment is isolated from one another.

Optionally, each compartment is integrally formed within a wall.

Optionally, the bioprocessing modules are arranged in an array of rows, or columns, or a combination of rows and columns.

In accordance with another aspect of the disclosure, there is provided a mobile facility comprising the modular bioprocessing system as described herein. Optionally, the mobile facility is provided as a trailer having wheels.

Optionally, the mobile facility comprises a clean room and a service room, wherein the clean room and the service room are separated by the modular bioprocessing system. In some examples, the clean room and the service room are separated by a wall, the modular bioprocessing system being integrally formed within the wall.

In accordance with yet another aspect of the disclosure, there is provided a modular bioprocessing system having a first side and a second side, the system comprising: a plurality of bioprocessing modules, each formed as a compartment on the first side, and each being independently configured to receive at least one biological handling container and perform one or more unit operations thereon and/or on contents therein; and a maintenance portion, formed on the second side, configured to provide one or more services to each bioprocessing module.

Optionally, the one or more services comprise at least one of: a fluid connection, an electrical connection, a gas connection, and a network connection.

Optionally, the second side comprises a control panel configured to control the one or more services provided to each bioprocessing module.

It is noted that embodiments of any modular bioprocessing system described herein may be combined with any other modular bioprocessing system described herein.

It is noted that the biological handling container may have any suitable form. For example, the biological handling container may comprise a top section, a base section arranged in parallel with the top section, and a flexible wall element extending between the top section and the base section. The flexible wall element may be compressible, so as to enable relative movement between the top section and the base section. The flexible wall element may have a plurality of folds, such as Z-folds. The biological handling container may be a flexible bag, a flexible bag retained within a rigid support, or a rigid container. The biological handling container may be substantially gas impermeable, substantially gas permeable or have a substantially gas permeable portion (such as a gas permeable flexible wall element and/or a gas permeable base section). Each biological handling container is independent of another, hence one biological handling container may assume the form of a compressible container, and another may assume the form of a rigid container, or any other combination contemplated herein.

In accordance with yet another aspect of the disclosure, there is provided a method of processing biological material comprising: loading a biological handling container into a first bioprocessing module of a plurality of bioprocessing modules; performing a first unit operation on the biological handling container and/or contents thereof within the first bioprocessing module; removing, via a robot, the biological handling container from the first bioprocessing module; moving, via the robot, the biological handling container to a second bioprocessing module of the plurality of bioprocessing modules; and performing a second unit operation on the biological handling container and/or contents thereof within the second bioprocessing module.

Optionally, the first unit operation and/or the second unit operation comprise introducing a population of cells into the biological handling container.

Optionally, the first unit operation and/or the second unit operation comprise introducing at least one of a cell culture medium, a growth factor, a viral vector, a non-viral vector or a bead-based reagent into the biological handling container.

Optionally, the first unit operation and/or the second unit operation comprise at least one of culturing, genetically modifying, stimulating, expanding, washing, separating, selecting, or harvesting cells contained within the biological handling container.

Optionally, the first unit operation and/or the second unit operation comprise storing the biological handling container under predetermined conditions.

Optionally, the first unit operation and/or the second unit operation comprise analyzing, centrifuging or electroporating the contents of the biological handling container.

Optionally, the step of loading a biological handling container into a first bioprocessing module of a plurality of bioprocessing modules comprises loading, via the robot, the biological handling container into the first bioprocessing module.

Any of the features or steps described herein in relation to one embodiment, aspect or example, of a bioprocessing system, a bioprocessing module, a biological handling container, a robot, a mobile facility or a method of processing biological material as described herein may be equally applicable to any other embodiment, aspect or example as described herein. Particularly, the bioprocessing system described herein may have any combination of bioprocessing modules as described herein.

### BRIEF DESCRIPTION OF THE DRAWINGS

The present disclosure is explained in more detail hereinbelow with reference to the figures:

FIG. 1 illustrates an overview of the components included in an exemplary modular bioprocessing system;

FIG. 2A illustrates a mobile facility according to an exemplary modular bioprocessing system;

FIG. 2B illustrates a robot and curved bank of bioprocessing modules according to an exemplary modular bioprocessing system;

FIG. 2C illustrates a robot on rails and a bank of bioprocessing modules according to an exemplary modular bioprocessing system;

FIG. 2D illustrates a robot on a rail and a bank of bioprocessing modules recessed in a wall included in an exemplary modular bioprocessing system;

FIG. 3 illustrates a clean room, with a robot and a bank of bioprocessing modules arrayed to encircle the robot;

FIG. 4 illustrates a bioreactor module;

FIG. 5A illustrates a first example of a biological handling container;

FIG. 5B illustrates a second example of a biological handling container;

FIG. 5C illustrates a cross section of the biological handling container of FIG. 5B;

FIG. 5D illustrates a third example of a biological handling container;

FIG. 5E illustrates a fourth example of a biological handling container;

FIG. 5F illustrates a fifth example of a biological handling container;

FIG. 6 illustrates a connector; and

FIG. 7 illustrates a partial section of a modular bioprocessing system.

### DETAILED DESCRIPTION

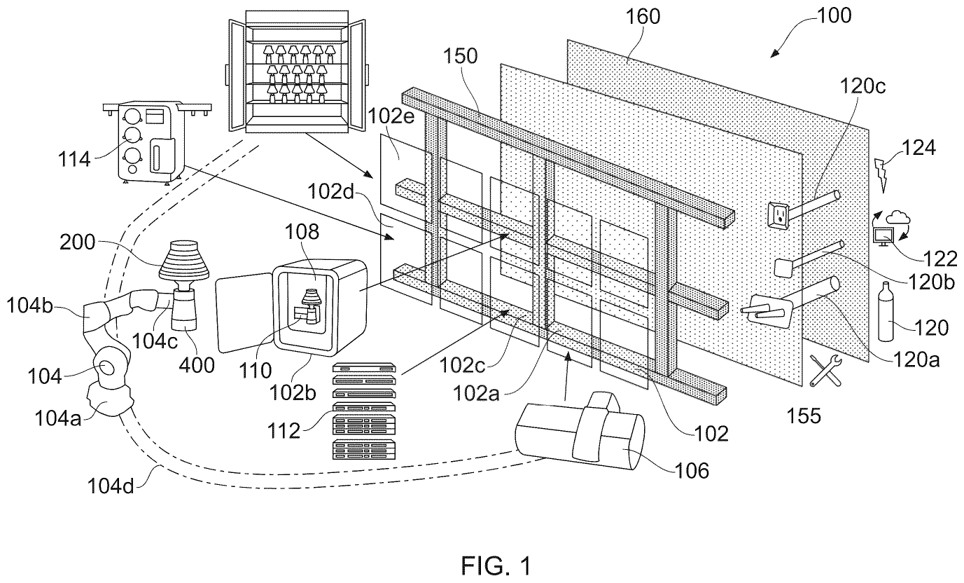

Referring to FIG. 1, there is provided a modular bioprocessing system 100 including a plurality of bioprocessing modules 102 and a robot 104. The bioprocessing modules 102 are provided within an array of rows and columns recessed into a wall. The bioprocessing modules 102 may be selected from any appropriate module as discussed herein, but for illustrative purposes, there is provided a biological analysis module 102a, including a biological analysis device 106 such as a pH meter, a microscope, a cell density meter or the like; a bioreactor module 102b, including an incubator unit 108 for housing a bioreactor (not shown) and including a dock 110; a server module 102c, including a server 112; a cell processing module 102d, including a spinning membrane filtration device 114; and a storage module 102e, including a plurality of docks (not shown). Generally, each bioprocessing module 102 may have means to receive a biological handling container 200, such as a dock 110 discussed above. The dock 110 may be provided as a holding or receiving mechanism and enable the interfacing of the biological handling container 200 and one or more processing devices to enable unit operations to be carried out on the biological handling container 200 and/or the contents thereof.

The robot 104 is provided with a base 104a, an articulating arm 104b, and a gripping portion 104c. The gripping portion 104c may be caused to actuate to enable gripping, or otherwise receiving, the biological handling container 200 during use. The robot 104, namely the base 104a, may be provided on a rail 104d to enable movement. Alternatively, the base 104a may be provided with wheels. The movement along the rail 104d or on wheels may be controlled remotely by a computer system.

The modular bioprocessing system 100 may be formed from a first side 150 and a second side 160. The first side 150 includes apertures, or a frame having apertures, into which each bioprocessing module 102 is disposed. The first side 150 may be regarded as the side from which the interior of each bioprocessing module 102 can be accessed. The second side 160 is provided at the opposing side of the modular bioprocessing system 100, and may be regarded as the side from which maintenance of each bioprocessing module 102 can occur. In particular, the second side 160 may enable access to various services, such as gas source 120, network connections 122 or electrical connections 124. There is also provided an interfacing portion 155 disposed between the first side 150 and the second side 160. The interfacing portion enables the services, i.e., gas, network and electrical connections, from the second side 160 to each bioprocessing module 102 on the first side 150. For example, gas connections 120a, network connections 120b and electrical connections 120c may be provided through the interfacing portion 155. The first side 150 may be provided in a clean room. The second side 160 may be provided in a service or maintenance room or corridor.

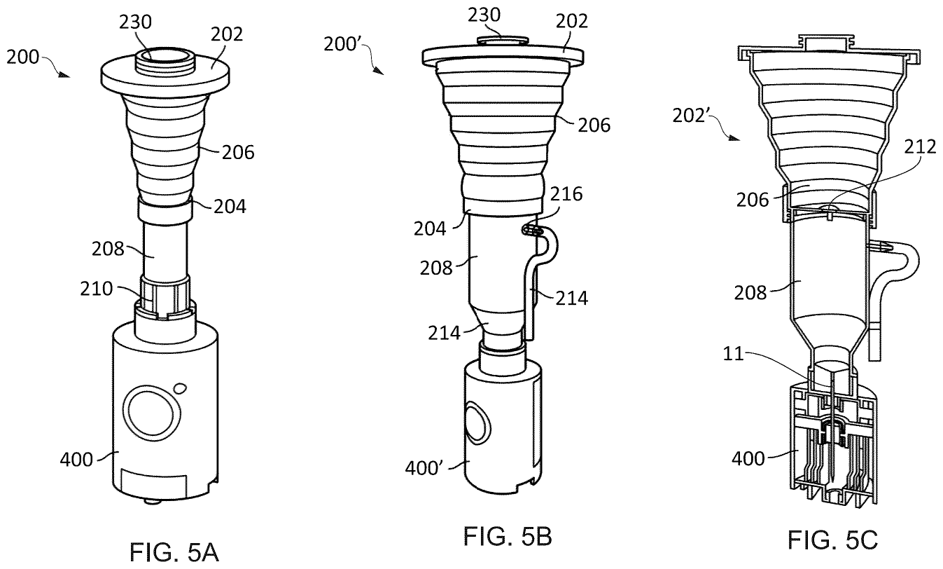

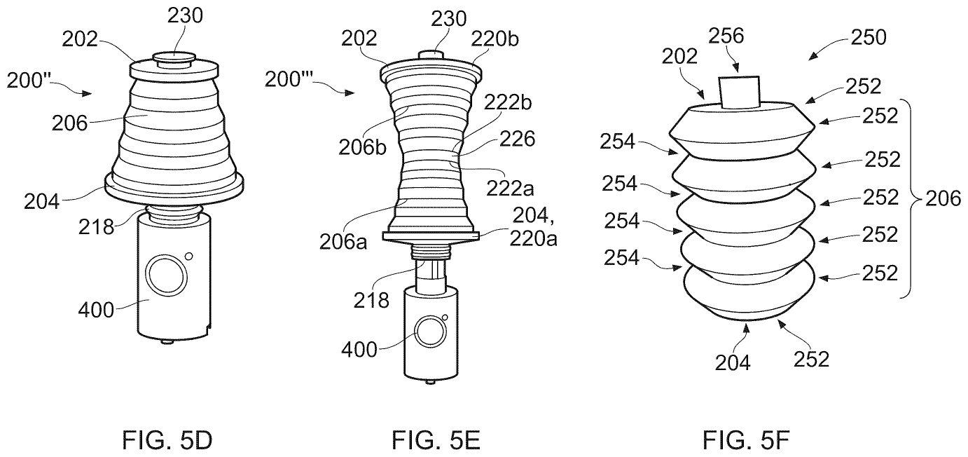

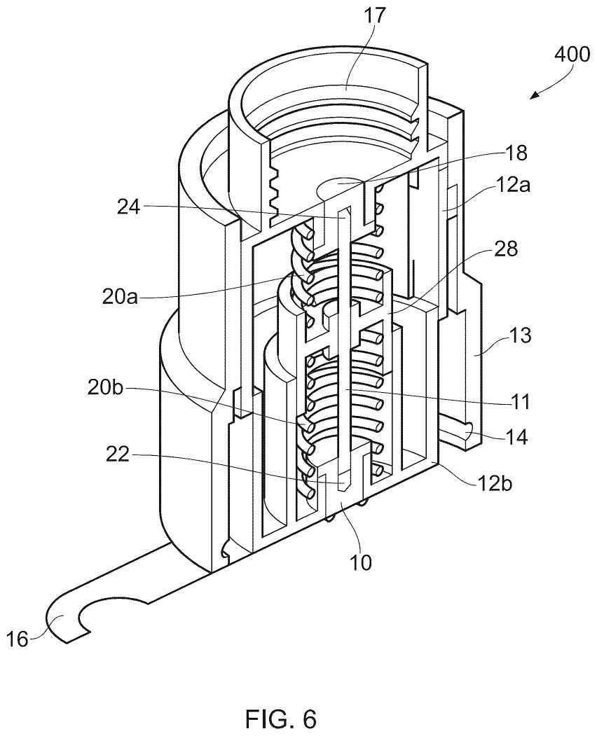

As illustrated in FIG. 1, the biological handling container 200 may include a container portion and a connector 400. The biological handling container 200 is described in more detail below and shown in FIG. 5A through 5F, but generally includes a top section and a bottom section that are compressible with respect to one another so as to cause dispensation. The connector 400 may be a needle-based connector as described below and shown in FIG. 6. The biological handling container 200 may be suitable for handling culture media, cellular material, viruses, or the like. The biological handling container 200 may be a bioreactor or an auxiliary container for inputting material into a bioreactor.

Referring further to FIG. 1, during use, the robot 104 retrieves a biological handling container 200 from a bioprocessing module 102, using the gripping portion 104c, and transports such biological handling container 200 to another, i.e., different, bioprocessing module 102. In this way, the biological handling container 200 can be transported in an automated manner between different bioprocessing modules 102, each handling a discrete unit operation in a bioproduct manufacturing process. For example, the robot 104 may retrieve a biological handling container 200 including culture media therein from storage module 102e, transport the biological handling container 200 from the storage module 102e to a bioreactor module 102b, and place such biological handling container 200 into a dock 110 within the bioreactor module 102b for dispensation by actuators within the bioreactor module 102b. The contents of the biological handling container 200−such as culture media, cellular material, viruses or the like—can be dispensed into a bioreactor received within the bioreactor module 102b. Once the operation is complete, the robot 104 may retrieve the used biological handling container 200 for transportation back to the storage module 102e, or for discarding in a bin or waste module (not shown).

As will be understood by those skilled in the art, the robot 104 may be controlled automatically by a control system through a network, such as a wireless network. Alternatively, the robot 104 may be controlled by a user interface or human-machine-interface provided on a mobile application, a tablet, or a screen.

The bioprocessing modules 102 may be any appropriate module suitable for use in the manufacture of a bioproduct, such as a cell and/or gene therapy product, more particularly a CAR-T product. Each of the bioprocessing modules 102 can be selected, independently, from any appropriate module, such as a bioreactor module formed as an incubator for housing a bioreactor therein; a biological analysis module including a pH meter, a cell counter, a cell seeding density meter, a flow cytometer, a polymerase chain reaction (PCR) device, a sterility analyzer, a media analyzer, a metabolite analyzer, a cell imaging device or a microscope; a preparation module for preparing material and/or dispensing material into the biological handling container; a cell selection module including a magnetically-activated cell selection device; a centrifuge module including a centrifuge device; an electroporation module including an electroporation device; a cell harvesting module including means to enable harvesting of cellular material from a container, such as a biological handling container, into a final form and/or package; or a storage module as described above. The disclosure is not intended to be limited by any means in respect of the module and/or devices utilized.

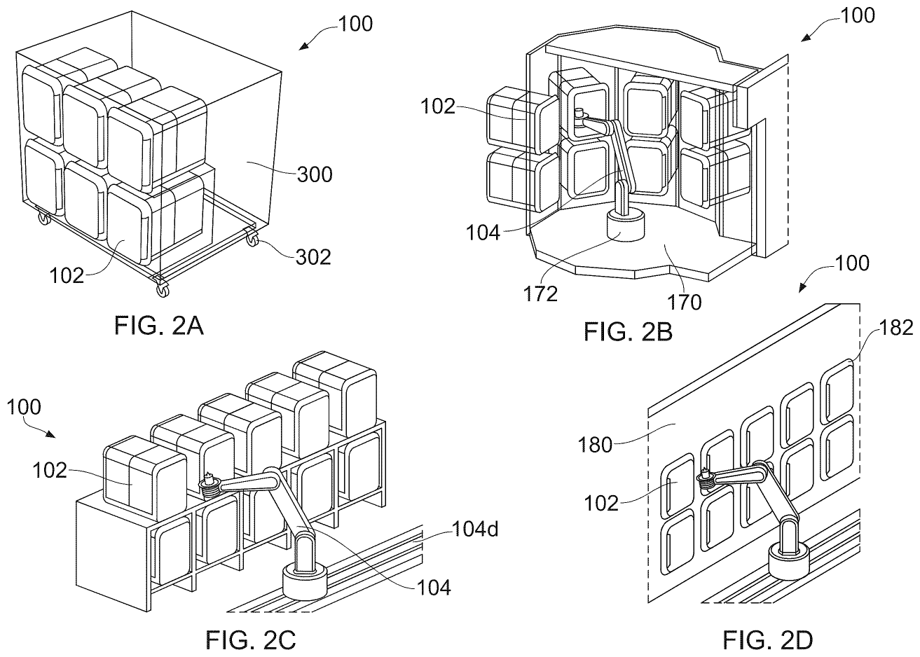

Referring now to FIG. 2A, there is provided one example of a modular bioprocessing system 100 provided in a mobile facility 300 with wheels 302 such that a user can move the mobile facility 300 from location to location. The mobile facility 300 includes the plurality of bioprocessing modules 102 therein. The mobile facility 300 can still include the features described in relation to FIG. 1, such various services including gaseous, electrical and network connections. In this way, the bioprocessing modules 102 can be provided with gases, electrical power and network connectivity during transportation from a first location to a second location or on-site whilst not being transported. The mobile facility 300 may include means for attachment to a vehicle, such as a tethering point, such that the mobile facility 300 can be transported on the road or within a larger facility such as a warehouse. The mobile facility 300 can be provided with a space for a robot (not shown), such that the bioprocessing module 102 and the robot can be transported simultaneously in a single trailer.

Referring now to FIG. 2B, there is provided one example of a modular bioprocessing system 100. In this example, the robot 104 is provided on a platform 170 and can rotate on a pedestal 172 about a longitudinal axis. The bioprocessing modules 102 are arrayed around the robot 104 in an arcuate manner such that the robot 104 can perform operations on each of the bioprocessing modules 102. This configuration increases the number of bioprocessing modules 102 that a single robot 104 can operate on whilst being moveable about a single point (i.e., without a rail). In FIG. 2B, the modular bioprocessing system 100 is provided with bioprocessing modules 102 stacked on top of one another, namely two bioprocessing modules 102 stacked on top of one another, but three or more bioprocessing modules 102 could be stacked on top of one another, with the robot 104 being able to vertically or telescopically translate and/or extend to reach higher bioprocessing modules 102.

Referring now to FIG. 2C, there is provided one example of a modular bioprocessing system 100, in which the bioprocessing modules 102 are arranged linearly in an array of rows and columns adjacent a robot 104 on a rail 104d as described in relation to FIG. 1. In this particular example, the rail 104d may extend in parallel to the array of bioprocessing modules 102.

Referring now to FIG. 2D, there is provided one example of a modular bioprocessing system 100, in which the bioprocessing modules 102 are recessed into a wall 180. The wall 180 may be a wall of a clean room, such that access to the interior of the bioprocessing modules 102 is limited to a defined number of users—i.e., those that only can access the clean room—thereby minimizing exposure to potentially harmful materials, such as viruses, fungi or bacterium. In this configuration, the side of the wall 180, which provides access to the bioprocessing module 102 is a first side of the overall modular bioprocessing system 100 (see FIG. 1) and the opposing side (not shown) is a second side of the overall modular bioprocessing system 100 (see FIG. 1). The first side allows the robot or designated human operatives access the interior of the bioprocessing modules 102. The second side includes various features that are more difficult to optimize for control by a robot and/or can be accessed by humans that are not required, trained or qualified to enter the clean room side. For example, the second side is configured for maintenance operations and includes gas sources, a network adaptor, and an electrical connection. These features are likely to require human intervention for maintenance, repair and the like, and it is preferable to not require a human operative to enter the clean room environment for such activities. The first side and the second side are isolated, for example, fluidly isolated i.e., not in fluid communication, such that no harmful materials can pass between them. In particular, a fluid tight, or hermetic, seal 182 is provided around each biological processing module to ensure isolation between the first side and the second side. In this way, the clean room is primarily occupied by robots during normal operation, with minimal human intervention, and the second side can be accessed by human operatives without the need to prepare to enter the clean room environment and/or be potentially exposed to harmful materials.

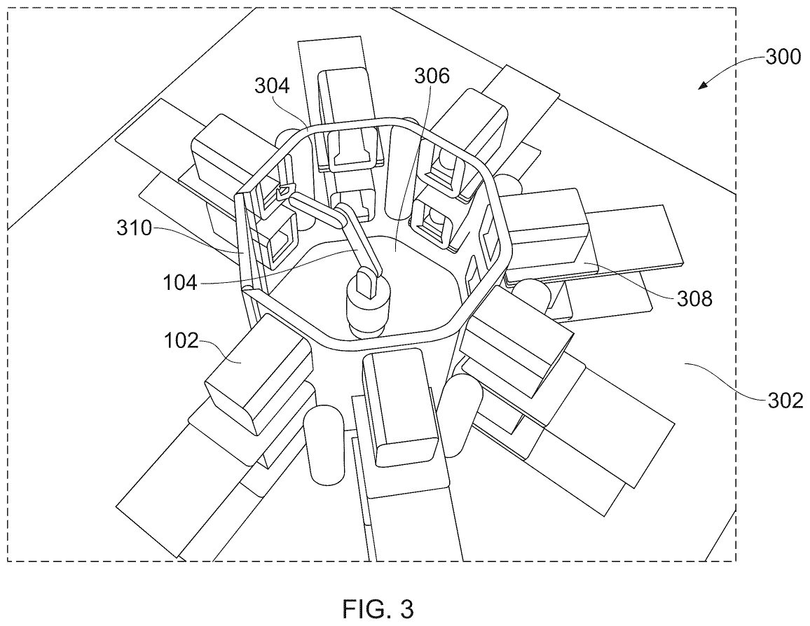

FIG. 3 illustrates an example of a clean room 300, including a circular array of bioprocessing modules 102, which are arrayed to encircle a robot 104. The bioprocessing modules 102 are arranged in this manner to maximize the utility of floor space within a first zone 302 of the clean room 300, as the robot 104 can access each of the bioprocessing module 102 without needing to be provided with rails. The first zone 302 is generally defined by an encircling wall 304 separating the first zone 302 from a second zone 306. The encircling 304 has apertures through which each bioprocessing module 102 is located and accessible by the robot 104 during use.

The first zone 302 may be thermally controlled or maintained to ensure the volume defined therein is maintained at substantially a constant temperature, for example, 37 degrees Celsius. Additionally or alternatively, the first zone 302 may have a controlled atmosphere, such that a content of gas is controlled or maintained therein. For example, the carbon dioxide content of the first zone 302 may be maintained at approximately 5% to approximately 10%.

Each bioprocessing module 102 is located on a slidable access tray 308, such that they can be each slidingly moved between a first position and a second position. In the first position, as shown in FIG. 3, each bioprocessing module 102 is disposed with an open end—i.e., for access via the robot 104—of each module 102 being disposed in the first zone 302. The open end may be sealed around its periphery to ensure a thermally and/or fluidly and/or hermetically sealed first zone 302, particularly sealed from, and with respect to, the second zone 306. In the second position (not shown), each bioprocessing module 102 is moved laterally away from the array of bioprocessing modules 102, such that the entirety of each bioprocessing module 102 is disposed within the second zone 306, thereby enabling ease of maintenance and/or service of each bioprocessing module 102.

The volume of the first zone 302 and the volume of each bioprocessing module 102 can be formed unitarily i.e., without the need for doors on each bioprocessing module 102. In this way, a single thermal and/or atmospheric environment is provided, without the need to control each bioprocessing module 102 individually. In other examples, each bioprocessing module 102 is provided with a door. Yet further, in some examples, each aperture, through which the bioprocessing modules 102 are located, includes a door so as to seal the first zone 302 prior to moving a bioprocessing module 102 to the second position. A user may access the first zone 302 through a door 310 within the wall 304 separating the first zone 302 and the second zone 306.

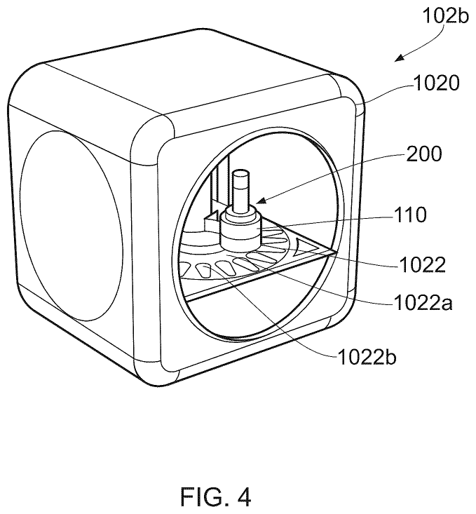

As an example of one such module for use in any of the systems described herein, there is provided a bioreactor module 102b as illustrated in FIG. 4. The bioreactor module 102b has the form of an incubator, with an enclosure 1020, which encloses an interior volume. Within the interior volume there is provided a dock 110 configured to hold a biological handling container 200. The dock 110 may be provided statically, i.e., immobile, or it may be moveable. In some examples, the dock 110 is rotatable about a central axis such that it can be indexed to a position at the front of the interior volume, close to an opening of the enclosure providing ease of access for the robot described above. Additionally or alternatively, a plurality of docks can be arrayed around this central axis and several biological handling containers can be located inside the interior volume. In such a case, the docks may be moveable like a carousel. For example, upon receiving a signal that a particular biological handling container is required for a unit operation, the relevant dock can be rotated, or indexed, into position at the front of the interior volume. Similarly, when an empty dock is required, the empty dock is rotated, or indexed, to the position at the front of the interior volume. As such, the movements of the robot can be simplified because only one location within the bioreactor module 102b needs to be accessed.

Additionally or alternatively, the bioreactor module 102b includes a moveable interface 1022. The moveable interface 1022 may move along a longitudinal axis, i.e., up and down in relation to the enclosure 1020, and/or the moveable interface 1022 may rotate around a central longitudinal axis. The moveable interface 1022 may be provided attached to a bioreactor (not shown) and serve to allow interfacing of the internal lumen of the bioreactor with another component, such as a container. The moveable interface 1022 includes an upper face 1022a with ports 1022b. The ports 1022b are constructed to interface and form an aseptic and fluid-tight, or hermetic seal with the biological handling container 200 via a connector (see FIG. 6), such that liquid, such as media and gas can be provided thereto. In particular, the ports 1022b may each be provided as a resealable septum seal. The ports 1022b may be provided with a further aseptic barrier, such as an aseptic paper seal, which is configured to mate with a corresponding aseptic barrier, such as a corresponding aseptic paper seal, which are removed before interfacing the biological handling container 200, via the connector (see FIG. 6), with the respective port 1022b.

FIG. 5A 5B, 5C, 5D and 5E illustrate various examples of biological handling containers 200, 200′, 200″, 200″′, 200″″ configured for use in the modular bioprocessing system 100. Each of the biological handling containers 200, 200′, 200″, 200″′, 200″″ include a top section 202, a bottom section 204 and a flexible and compressible sidewall 206, which is arranged between the top section 202 and the bottom section 204. Each biological handling container 200, 200′, 200″, 200″′, 200″″ includes a connector 400, particularly a needle-based connector, coupled to a distal end thereof, which shall be described in more detail below.

Generally, the connector 400 can act as both an inlet and outlet for fluid, including liquid, gases, suspensions, slurries, gels or the like, between each biological handling container 200, 200′, 200″, 200″′, 200″″ and another container or component.

As shown in FIG. 5A, the bottom section 204 of biological handling container 200 is coupled to, and partially encloses, a vial 208, which includes an internal volume for holding fluid. The fluid in the internal volume can be dispensed via the connector 400. The compressible sidewall 206 above the vial 208 is flexible and contains a plunger (not shown) which can move reciprocally within the vial 208, such that the volume of the vial 204′ can be changed by applying a compressive force to the top section 202 and/or the bottom section 204. The biological handling container 200 shown in FIG. 5A additionally includes a mounting collar 210. The mounting collar 210 includes a feature that allows it to be removably attached to the connector. One example feature is an external thread for connecting the mounting collar to a threaded portion of the connector 400. Additionally or alternatively, the mounting collar 210 may push fit onto the connector 400 and include an O-ring or other elastomeric member to increase the holding force. It will be appreciated that other connection mechanisms may be provided to the mounting collar 210 and the connector 400 to provide a connection mechanism.

The biological handling container 200 illustrated in FIG. 5A may be configured and arranged to store and/or dispense viruses, magnetically-activated reagents, or magnetic beads.

As shown in FIG. 5B and FIG. 5C, the bottom section 204 of the biological handling container 200′ is coupled to, and partially encloses, a vial 208, which includes an internal volume for holding fluid, similar to that described above in respect of FIG. 5A. However, the vial 208 of FIG. 5B is not provided with a plunger, but instead compression of the top section 202 with respect to the bottom section 204 causes contents within the vial 208 to be dispensed, due to an air drive mechanism (i.e., a positive air pressure being created). In particular, there is provided a one-way valve 212 between the bottom section 204 and the vial 208 to enable fluid, such as air, contained within the volume defined by the top section 202, the bottom section 204 and the compressible sidewall 206 to pass in one direction into the vial 208 and urge fluid form the vial 208 through a hollow needle 11 of the connector 400. The biological handling container 200′ shown in FIG. 5B additionally includes a feed tube 214, which can be used to add or remove fluid to and from the vial 204′ via a spigot 216. After use the feed tube 214 can be detached or sealed off, for example, plugged or welded shut. Any part of the feed tube 214 remaining attached to the biological handling container 200′ can be clipped to the vial 204. The biological handling container 200′ additionally includes a funnel portion 216 extending from beneath the vial 204′, which tapers inwardly such that fluids are directed toward the connector 400′ in use.

The biological handling container 200′ illustrated in FIGS. 5B and 5C may be configured and arranged to store and/or dispense cell culture media, or cellular starting material.

As shown in FIG. 5D, the biological handling container 200″′ does not include a vial in comparison to the examples of FIGS. 5A and 5B. Instead, the volume defined by the top section 202, the bottom section 204 and the compressible sidewall 206 is arranged to hold a fluid for dispensation. In particular, the top section 202, the bottom section 204 and the compressible sidewall 206 define a frustoconical container. The bottom section 204 includes a screw threaded portion 218 for coupling the biological handling container 200″′ to the connector 400. During use, the top section 202 is compressed with respect to the bottom section 204 to cause a dispensation of fluid from within the volume defined therein.

The biological handling container 200″ illustrated in FIG. 5D may be configured and arranged to store and/or dispense cell culture media, or cellular starting material.

As shown in FIG. 5E, the biological handling container 200″″ is substantially the same as that described in FIG. 5D. In this example, the biological handling container 200″″ a first collapsible side wall 206a and a second collapsible side wall 206b. Each of the first and second collapsible side walls 206a, 206b has a frustoconical form. Each of the first and second collapsible side walls 206a, 206b has a first end 220a, 220b and a second end 222a, 222b, the first ends 220a, 220b, being larger than the second ends 222a, 222b. The first and second collapsible side walls 206a, 206b are joined end-to-end such that they define a single internal volume, or lumen, and collapse in the same direction. In the illustrated example, the second ends 222a, 222b (i.e., the smaller ends) are joined to each other. The first end 220a of the first collapsible side wall 206a is provided with the bottom section 204 and is attached to the connector 400 via a screw threaded portion 218. The first end 220b of the second collapsible side wall 206b is provided with the top section 202. Accordingly, from the bottom section 204, the first collapsible side wall 206a tapers inwards to a narrower waist 226 and then the second collapsible side wall 206b tapers outwards to the top section 202. The bottom section 204 also includes a screw threaded portion 218 to enable coupling to the connector 400.

The biological handling container 200″″′ illustrated in FIG. 5E may be configured and arranged to store and/or dispense cell culture media, or cellular starting material.

As shown in FIG. 5F, the biological handling container 250 may be provided without a connector compared to the previous examples. In this example, the biological handling container 250 includes a top section 202, a bottom section 204 and a flexible and compressible sidewall 206. As illustrated, and as noted in the other examples, the flexible and compressible side wall has a number of annular rigid sections 252 laterally arranged in parallel to both the top section 202 and the bottom section 204. The annular rigid sections are interleaved with a deformable region 254 to enable compression. The biological handling container is also provided with a port 256 at the top section 202, although this may be a screw threaded portion of the top section 202 in other examples.

The biological handling container 250 illustrated in FIG. 5F may be a bioreactor.

The biological handling containers 200, 200′, 200″, 200″′, 200″″, 250, may include an on-board network adaptor and may also include on-board control and analysis devices such as a battery, a sensor, a heater, a pH meter, a thermometer etc., such that constant monitoring of the contents is possible. The network adaptor can transmit a signal indicative of the status of the on-board control and analysis devices, including the temperature, pH of the media, charge level of the battery, etc.

It is noted that, as an optional feature, each of the biological handling containers 200, 200′, 200″, 200″′, 200″″ of FIGS. 5A, 5B, 5C, 5D and 5E has an engaging feature 230, which is adapted for an actuator, and which may be part of the robot 104 or part of a biological processing module to engage and cause dispensation of the contents of the biological handling containers 200, 200′, 200″, 200″′, 200″″ through compression of the top section 202 with respect to the bottom section 204.

The biological handling containers 200, 200′, 200″, 200′″, 200″″, 250 as discussed above may also include a designation feature, which may be an RFID tag, an NFC tag, a barcode or a QR code, or any combination thereof. The robot 104, preferably the gripping portion 104c of the robot has a complimentary reader, such as a scanner, which is used to detect which biological handling container 200, 200′, 200″, 200″′, 200″″, 250, is being collected by the robot 104. In this way, if a biological handling container 200, 200′, 200″, 200″′, 200″″, 250, has been moved from its known location, for example by an operative, and replaced in a different dock to the location previously stored on the network, the reader can confirm which biological handling container 200, 200′, 200″, 200″′, 200″″, 250 from a set of biological handling containers 200, 200′, 200″, 200″′, 200″″, 250 has been collected. A microprocessor and associated memory can be used to designate each dock 110 with an identity and known location and when a biological handling container 200, 200′, 200″, 200″′, 200″″, 250 is docked the memory can be updated with information to confirm its presence or absence. The robot can scan the designation feature each time it interacts with a biological handling container 200, 200′, 200″, 200″′, 200″″, 250 such that the correct biological handling container 200, 200′, 200″, 200″′, 200″″, 250 is collected each time. If the robot is performing an operation to collect a biological handling container 200, 200′, 200″, 200″′, 200″″, 250 and detects that it is missing or incorrectly designated, a signal indicating an error can be provided to an operative via the network adaptor.

The biological handling containers 200, 200′, 200″, 200″′, 200″″ of FIG. 5A through 5E include a connector 400, which is constructed to compliment the docks 110 and/or the moveable interface 1022 of the bioreactor module 102b (see FIG. 4). The connector 400 may include one or more docking features that additionally includes a connection element for receiving gas from the gas source 120, a connection element for electrical connection to the electrical connector 124 and a connection element to a network terminal for connection to the network. In this way, when the biological handling container 200, 200′, 200″, 200″′, 200″″, namely the connector 400 thereof, is docked it can transmit signals indicative of the status of the medium, cells, temperature, battery charge etc. If power, additional gas or fluids are required a signal can be sent via the on-board network adaptor to the network adaptor 122. The connector 400 is a needle-based connector having a hollow needle 11 arranged to pierce a septum seal of both the connector (as described below in relation to FIG. 6) and also a septum seal disposed at either the distal end of the vial (FIGS. 5A, 5B, 5C) or of the bottom section 204 (FIGS. 5D, 5E).

FIG. 6, illustrates a cross-sectional view of the connector 400, which is used to connect the biological handling container 200 to another component in use, such as the moveable interface 1022 of the bioreactor module 102b (see FIG. 4). The connector 400 comprises a housing having an upper housing portion 12a and a lower housing portion 12b. The housing extends along a longitudinal axis between a distal end and a proximal end. The upper housing portion 12a may be axially moveable, or slidable, with respect to the lower housing portion 12b, as will be described further below.

The housing includes a threaded portion 17 at its distal end for connecting to a corresponding threaded portion of the vial. The threaded portion 17 is formed on the upper housing portion 12a. As will be clear to the skilled person, the housing may be provided without the threaded portion 17, and instead be provided with another suitable connection mechanism for connecting to a portion of the vial.

The connector 400 also includes a connector portion at its proximal end for connecting to the bioreactor module 102b of the moveable interface 1022. The connector portion may be a groove 14, configured to receive one or more protrusions extending from the moveable interface 1022. Alternatively, the connector 400 may comprise a threaded portion or other connector portion for connecting to the moveable interface.

The connector 400 includes a first septum seal 18 disposed at the distal end of the housing, and a second septum seal 10 disposed at the proximal end of the housing. The first septum seal 18 includes a substantially planar, i.e., flat, pierceable surface facing outwardly at the distal end. The second septum seal 10 includes a generally annular portion, extending outwardly at the proximal end, enclosing a substantially planar, i.e., flat, pierceable surface facing outwardly at the proximal end. The housing further includes a hollow needle 11 that is biasedly mounted within the housing. The hollow needle 11 is generally coaxially aligned with the longitudinal axis. The hollow needle 11 includes a first end 24, facing the first septum seal 18, and a second end 22, facing the second septum seal 10. The first end 24 is configured to be able to pierce the first septum seal 18, in use, and the second end 22 is configured to be able to pierce the second septum seal 10, in use. The first septum seal 18, the second septum seal 10, or both the first and second septum seal 18, 10 may optionally be provided with a removable aseptic paper seal 16.

The hollow needle 11 is mounted within the housing through a collar 28 that is spring-biased by a first spring 20a and a second spring 20b. In other embodiments, the hollow needle 11 may be mounted in another suitable manner, for example, the hollow needle 11 may be statically mounted, i.e., such that it does not move, and the housing may be moveable about the hollow needle 11. The first spring 20a acts between the distal end of the housing and the collar 28. The second spring 20b acts between the proximal end of the housing and the collar 28. In this way, the first spring 20a provides a first biasing force to the hollow needle 11, via the collar 28, in a direction toward the proximal end of the housing, and the second spring 20b provides a second biasing force to the hollow needle 11, via the collar 28, in a direction toward the distal end of the housing.

The connector 400 further includes an actuating mechanism for causing the hollow needle 11 to pierce the septum seals 10, 18. By piercing the first and second septum seals 10, 18 the hollow needle 11 creates a fluid path between the distal end and the proximal end of the connector 400, and so during use creates a fluid connection between the vial 204′, 208′ of the biological handling container and the bioreactor module 102b.

In the example illustrated in FIG. 6 the actuating mechanism includes an outer sleeve 13 that is arranged to collapse the upper housing portion 12a with respect to the lower housing portion 12b. The outer sleeve 13 is rotatable with respect to the housing about the central longitudinal axis of the housing. For example, one of the outer sleeve 13 and the housing may include a helical groove, and the other of the outer sleeve 13 and housing may include a protrusion that engages the groove such that when the upper housing portion 12a collapses with respect to the lower housing portion 12b the outer sleeve 13 is rotated.

It is noted that, whilst an actuating mechanism including an outer sleeve and springs biasing the needle and/or the housing are illustrated, other actuating mechanisms are equally contemplated. The connector 400 illustrated is merely provided as an example of a means for providing connection between components of the system.

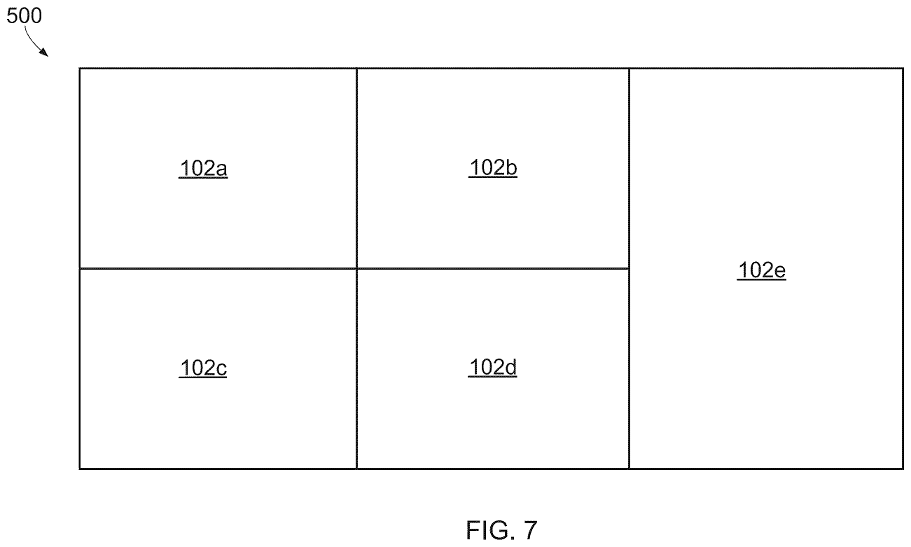

Referring now to FIG. 7, a section of a modular bioprocessing system 500 and five different bioprocessing modules are shown. Each bioprocessing module is specialized for a different purpose with the inclusion of specific components. For example, the top left module may be an analysis module 102a and contains an analysis device. One possible analysis device may be an automated cell counter, which captures images indicative of the cell density within the biological handling container 200, such that by taking regular measurements a growth rate can be established. Alternatively or additionally, the analysis module may include a pH meter, a cell counter, a cell seeding density meter, a flow cytometer, a polymerase chain reaction device, a sterility analyzer, a media analyzer, a metabolite analyzer or a cell imaging device.

The top center bioprocessing module may be a bioreactor module 102b as shown in FIG. 4 and described above.

The bottom left module may be a server module 102c and includes a server 112, which provides computing services including digital memory, control of the robot and network capabilities including internet access. The server can process signals indicative of the state and locations of the docks 110 and biological handling containers 200 as well as the temperature within the modular bioprocessing system 100, remaining stocks of gas, empty biological handling containers, media and various supplies required for media, consumables, etc. These signals can be processed to generate warnings, such as a low supply warning for gas, a low temperature warning or a door open warning. The skilled person will understand that there are various other warnings the server may be required to generate. The server may also generate error messages, for example, if a biological handling container is mislocated or dropped or if maintenance is required for a specific component, such as a heater.

The cell processing module 102d is a preparation module, which comprises a dock 110 as well as at least one preparation device 114. The preparation device may be a device for preparing media to be added to biological handling containers 200, 200′, by warming, mixing and decanting the media. Alternatively or additionally, the preparation device may be a spinning membrane filtration device, a centrifuge, a water bath, a flow cytometer or a water purification system. The skilled person will recognize that there are various other preparation devices that can be used in this module and that this is not an exhaustive list.

Each of the modules may be sized differently to be an appropriate size for their function, but preferably there will be a standard 1×1 size and larger modules will be sized to allow tessellation of the modules such that a larger module is 1×2 or 2×2 or 3×1 or 4×4 relative to the standard size. By way of non-limiting example, the right most module is a 1×2 sized storage module 102e. The storage module 102e may contain a number of docks 110 for biological handling containers 200, 200′, 200″, 200″′, 200″″, 250. The biological handling containers 200, 200′, 200″, 200″′, 200″″, 250 can be held in the storage module 102e for a whilst a biological material is grown within. Regular operations may be performed on the biological handling containers 200, 200′, 200″, 200″′, 200″″, 250 to ensure that the biological material is growing at the expected rate. Alternatively or additionally, the biological handling containers 200, 200′, 200″, 200″′, 200″″, 250 may provide signals to the server 112 indicative of the need for an operation to be performed, such as analysis by an analysis device or preparation of the biological material for long term storage or delivery. Alternatively, the storage module may be refrigerated to maintain a temperature of around 4 degrees Celsius or −20 degrees Celsius or −80 degrees Celsius for the storage of cell culture media, and other components for future use.

Certain terminology is used in the following description for convenience only and is not limiting. The words ‘right’, ‘left’, ‘lower’, ‘upper’, ‘front’, ‘rear’, ‘upward’, ‘down’, ‘downward’, ‘above’ and ‘below’ designate directions in the drawings to which reference is made and are with respect to the described component when assembled and mounted (e.g., in situ). The words ‘inner’, ‘inwardly’ and ‘outer’, ‘outwardly’ refer to directions toward and away from, respectively, a designated centerline or a geometric center of an element being described (e.g., central axis), the particular meaning being readily apparent from the context of the description.

Further, as used herein “clean room,” “cell culture lab,” “sterile environment” and like terms are intended to relate to a room in which outside contaminants can cause negative outcomes for cell culture, and in which the interface layer is preferably located, whereas the maintenance layer should be accessible from outside the clean room (or similar).

Further, as used herein, the terms ‘connected’, ‘attached’, ‘coupled’, ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, ‘first’, ‘second’, ‘third’ etc., merely indicate that different instances of like objects are being referred to and are not intended to imply that the objects so described must be in a given sequence, either temporally, spatially, in ranking or in any other manner.

Through the description and claims of this specification, the terms ‘comprise’ and ‘contain’, and variations thereof, are interpreted to mean ‘including but not limited to’, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality, as well as, singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract or drawings), or to any novel one, or any novel combination, of the steps of any method or process so disclosed.

1. A modular bioprocessing system, comprising:
a plurality of bioprocessing modules, each being independently configured to receive at least one biological handling container and perform one or more unit operations thereon and/or on contents therein; and
a robot configured to move the, or each, biological handling container between each of the plurality of bioprocessing modules.

2. The modular bioprocessing system of claim 1, wherein each bioprocessing module is immobile.

3. The modular bioprocessing system of claim 1, wherein at least one of the plurality of bioprocessing modules is a bioreactor module configured to maintain suitable conditions for cell culture, and optionally wherein the bioreactor module comprises an incubator configured to substantially maintain an internal atmosphere and/or temperature, and optionally wherein the bioreactor module comprises a first dock that is configured to receive a first biological handling container, and a second dock that is configured to receive a second biological handling container, and further optionally wherein a first biological handling container and a second biological handling container are received in the respective first dock and second dock, and still further optionally wherein the first biological handling container is a bioreactor and the second biological handling container is an auxiliary container.

4. The modular bioprocessing system of claim 1, wherein at least one of the plurality of bioprocessing modules is a biological analysis module, and optionally wherein the biological analysis module comprises at least one of: a pH meter, a cell counter, a cell seeding density meter, a flow cytometer, a polymerase chain reaction device, a sterility analyzer, a media analyzer, a metabolite analyzer, a cell imaging device and a microscope.

5. The modular bioprocessing system of claim 1, wherein at least one of the plurality of bioprocessing modules is a preparation module, and optionally wherein the preparation module is configured to prepare and/or process at least one of: cell culture media, growth factors, viral vectors, non-viral vectors and bead-based reagents.

6. The modular bioprocessing system of claim 5, wherein the preparation module is configured to dispense at least one of: cell culture media, growth factors, viral vectors, non-viral vectors and bead-based reagents into the biological handling container.

7. The modular bioprocessing system of claim 1, wherein one or more of the plurality of bioprocessing modules each further comprise a biological handling container.

8. The modular bioprocessing system of claim 7, wherein the biological handling container is any of a bioreactor, a consumable, an auxiliary container, a sampling container, or a vacutainer.

9. The modular bioprocessing system of claim 7, wherein the biological handling container comprises a flexible wall element, and optionally wherein the flexible wall element is compressible, or alternatively, the flexible wall element comprises a plurality of folds.

10. The modular bioprocessing system of claim 1, wherein at least one of the plurality of bioprocessing modules is selected from: a cell selection module such as a magnetic-activated cell selection module, a centrifuge module, a washing module, an electroporation module, a cell harvesting module, a formulation module, and a storage module.

11. The modular bioprocessing system of claim 1, wherein at least one of the plurality of bioprocessing modules comprises a device for reading a designation feature on the biological handling container, optionally wherein the designation feature is one or more of an RFID tag, an NFC tag, a barcode or a QR code.

12. The modular bioprocessing system of claim 1, further comprising a microprocessor configured to:
receive a signal from at least one of the plurality of bioprocessing modules; and
in response to receiving the signal from at least one of the plurality of bioprocessing modules, generate and send a signal to the robot such that the robot is caused to move the biological handling container from a first bioprocessing module to a second bioprocessing module.

13. The modular bioprocessing system of a claim 1, wherein each bioprocessing module is formed as a compartment.

14. The modular bioprocessing system of claim 13, wherein the system comprises a first side and a second side, each compartment formed on the first side and a maintenance portion formed on the second side, the maintenance portion comprising at least one of: a fluid connector, a fluid reservoir, an electrical connector, a gas connector, a gas source, a computer, a network adaptor and a control panel.

15. The modular bioprocessing system of claim 13, wherein an interior of each compartment is isolated from an interior of another compartment.

16. The modular bioprocessing system of claim 13, wherein each compartment is integrally formed within a wall.

17. The modular bioprocessing system claim 1, wherein the bioprocessing modules are arranged in an array of rows and/or columns.

18. A mobile facility comprising a modular bioprocessing system, comprising:
a plurality of bioprocessing modules, each being independently configured to receive at least one biological handling container and perform one or more unit operations thereon and/or on contents therein; and
a robot configured to move the, or each, biological handling container between each of the plurality of bioprocessing modules.

19. A method of processing biological material comprising:
loading, a biological handling container into a first bioprocessing module of a plurality of bioprocessing modules;
performing a first unit operation on the biological handling container and/or contents thereof within the first bioprocessing module;
removing, via a robot, the biological handling container from the first bioprocessing module;
moving, via the robot, the biological handling container to a second bioprocessing module of the plurality of bioprocessing modules; and
performing a second unit operation on the biological handling container and/or contents thereof within the second bioprocessing module.

20. The method of claim 19 wherein the first unit operation and/or the second unit operation comprise introducing a population of cells into the biological handling container.

21. The method of claim 19, wherein the first unit operation and/or the second unit operation comprise introducing at least one of a cell culture medium, a growth factor, a viral vector, a non-viral vector or a bead-based reagent into the biological handling container.

22. The method of claim 19, wherein the first unit operation and/or the second unit operation comprise at least one of culturing, genetically modifying, stimulating, expanding, washing, separating, selecting, or harvesting cells contained within the biological handling container.

23. The method of claim 19, wherein the first unit operation and/or the second unit operation comprise storing the biological handling container under predetermined conditions.

24. The method of claim 19, wherein the first unit operation and/or the second unit operation comprise analyzing, centrifuging or electroporating the contents of the biological handling container.

25. The method of claim 19, wherein the step of loading a biological handling container into a first bioprocessing module of a plurality of bioprocessing modules comprises loading, via the robot, the biological handling container into the first bioprocessing module.
